(19) United States
(12) Patent Application Publication
(10) Pub. No.: US 2014/0288481 A1
(43) Pub. Date: Sep. 25, 2014

(54) TRANSDERMAL PATCH WITH NON-WATER SOLUBLE ADHESIVE AND ANESTHETIC

(71) Applicant: G. Chad MORGAN, Chattanooga, TN (US)

(72) Inventor: G. Chad MORGAN, Chattanooga, TN (US)

(21) Appl. No.: 14/212,610

(22) Filed: Mar. 14, 2014

Related U.S. Application Data
(60) Provisional application No. 61/787,612, filed on Mar. 15, 2013.

(51) Int. Cl. A61K 9/70; A61N 1/30; A61K 31/167

(52) U.S. Cl. CPC A61K 9/7023; A61K 31/167; A61N 1/303
USPC 604/20; 424/449; 514/626

(57) ABSTRACT

Embodiments associated with a transdermal delivery device are described. In one embodiment, a transdermal delivery device comprises material coated on one surface with a mixture of a non-water soluble adhesive and an anesthetic.

### CROSS REFERENCE TO RELATED APPLICATIONS

This patent disclosure claims the benefit of U.S. Provisional Patent Application Ser. No. 61/787,612 entitled “Transdermal Patch With Non-Water Soluable Adhesive and Lidocaine,” filed on Mar. 15, 2013, which is hereby wholly incorporated by reference in its entirety.

### BACKGROUND

Drug delivery is a growing area in the pharmaceutical industry since it is an alternative to injections. Transdermal drug delivery systems feature noninvasive delivery of medication through a patient's skin. Medication can pass through the skin's protective barrier in one of two ways: passive absorption and active penetration.

A transdermal patch is used as a common way of passive drug delivery. For example, a patch is coated with a selected medicine or drug and attached to a patient's skin. Once applied to the patient's skin, the coated patch delivers a defined dose of medication over a period of time. The drug is absorbed through the skin and into the bloodstream. The nicotine patch is one example, but other common uses include pain management, motion sickness, or hormone replacement therapy.

A transdermal patch is typically attached to a patient by a water-soluble adhesive composition that is coated on the patch. However, mixing the adhesive and the medicine/drug composition deteriorates the adhesive properties of the water-soluble adhesive, which is undesirable. In prior techniques to address this issue, the adhesive was separated from the medicine where the adhesive is on one portion of the patch and the medicine is on a different portion of the patch.

### SUMMARY

In one embodiment of the disclosure, a transdermal delivery device comprises material coated on one surface with a mixture of a non-water soluble adhesive and an anesthetic.

In another embodiment, the transdermal delivery device comprises an electrode combined with the transdermal delivery device.

### BRIEF DESCRIPTION OF THE DRAWINGS

The accompanying drawings, which are incorporated in and constitute a part of the specification, illustrate various devices, methods, and other embodiments of the disclosure. It will be appreciated that the illustrated element boundaries (e.g., boxes, or other shapes) in the figures represent one embodiment of the boundaries. In some embodiments one element may be designed as multiple elements or that multiple elements may be designed as one element. In some embodiments, an element shown as an internal component of another element may be implemented as an external component and vice versa. Furthermore, elements may not be drawn to scale.

FIG. 1 illustrates one embodiment of a transdermal drug delivery device.

FIG. 2 illustrates another embodiment of a transdermal device combined with an electrode.

FIG. 3 illustrates the electrode of FIG. 2 separated from the patch material.

FIG. 4 illustrates the patch material of FIG. 2 separated from the electrode.

### DETAILED DESCRIPTION

Various embodiments of a device are disclosed for delivering a substance transdermally. In one embodiment, a drug delivery device is disclosed that provides improved adhesive properties in combination with an anesthetic. Lidocaine is one type of topical anesthetic that can be used to relieve pain and/or itching in an area when applied to skin or other tissue. However, lidocaine has damaging effects on the adhesive properties of water-based or water-soluble adhesives that are used to hold a transdermal patch on the skin. This causes the patch to easily detach from the skin or simply fall off prematurely.

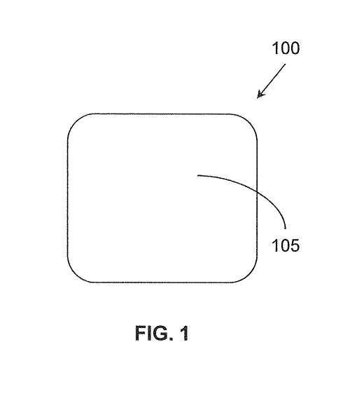

With reference to FIG. 1, in one embodiment, a transdermal drug delivery device 100 is illustrated. The device 100 is a skin attachable patch that is made with one surface 105 coated with a mixture of a non-water soluble adhesive and lidocaine. For example, the mixture is a combined composition of an adhesive composition and a lidocaine composition. Unlike water soluble adhesives, a non-water soluble adhesive is much less affected by the damaging effects of being exposed to lidocaine or other anesthetic. Using a non-water soluble (non-aqueous based) adhesive, the transdermal patch 100 has improved adhesive properties (when combined with an anesthetic) that are more consistent over time.

The consistent adhesion allows for removal of the patch 100 from the skin at the patient's discretion rather than prematurely detaching or falling off the skin due to loss of adhesive properties. The consistent adhesion also creates a longer term and consistent delivery of lidocaine or other selected anesthetic (e.g., benzocaine, butamben, dibucaine, lidocaine, oxybuprocaine, pramoxine, proparacaine, proxymetacaine, tetracaine) that is coated on the surface 105 and applied to the skin.

In one embodiment, the non-water soluble adhesive is polyisobutylene, also known as “PIB” or polyisobutene, (C₄H₈)ₙ. PIB is the homopolymer of isobutylene, or 2-methyl-1-propene, on which butyl rubber is based.

The polyisobutylene adhesive can be combined with lidocaine (or other selected anesthetics) in a variety of ratios and weights. For example, the polyisobutylene adhesive may be coated on the patch at about a weight of 7 mg/cm² for normal adhesive strength or can be increased (e.g., to about 10 mg/cm², which would make it very difficult to remove from skin once attached). In one embodiment, the adhesive is a gel-like substance.

In one embodiment, the lidocaine can be applied/combined with the adhesive as a 1% to 10% solution. Under current U.S. laws at the time of filing the present application, a 4% or less solution of lidocaine is non-prescription strength that can be sold over-the-counter. In other embodiments, other selected anesthetics at varying solution percentages can be applied and combined to form the adhesive-medicine composition.

In one embodiment, the transdermal drug delivery device is a cloth patch that is coated with the mixture of the non water-soluble adhesive and lidocaine (or other selected anesthetics). The cloth is breathable (nonocclusive), non-woven, and/or flexible. The patch may be configured as an adhesive bandage or tape (e.g., pain tape, elastic therapeutic tape, kinesiology tape, etc.). In another embodiment, the patch may be made from foam material (e.g., neoprene) or other type of material. In general, the patch is a substrate with at least one surface including the adhesive plus anesthetic composition coated thereon.

In other embodiments, the patch material can be preconfigured in various shapes and sizes to provide extremity specific patches. In a tape form, the tape can be configured in pre-cut strips or in a roll that can be cut by a user. In one embodiment (see FIG. 4), the patch 205 or tape may include notches 235 or perforations in side edges so as to improve the patch's ability to bend and stick to curved parts of a body.

In one embodiment, the composition of the polyisobutylene adhesive may include other compounds such as a vasodilator (e.g., menthol, 1% menthol, methyl salicylate, etc.), and may or may not include lidocaine (or other selected anesthetics).

### Electrode Embodiment

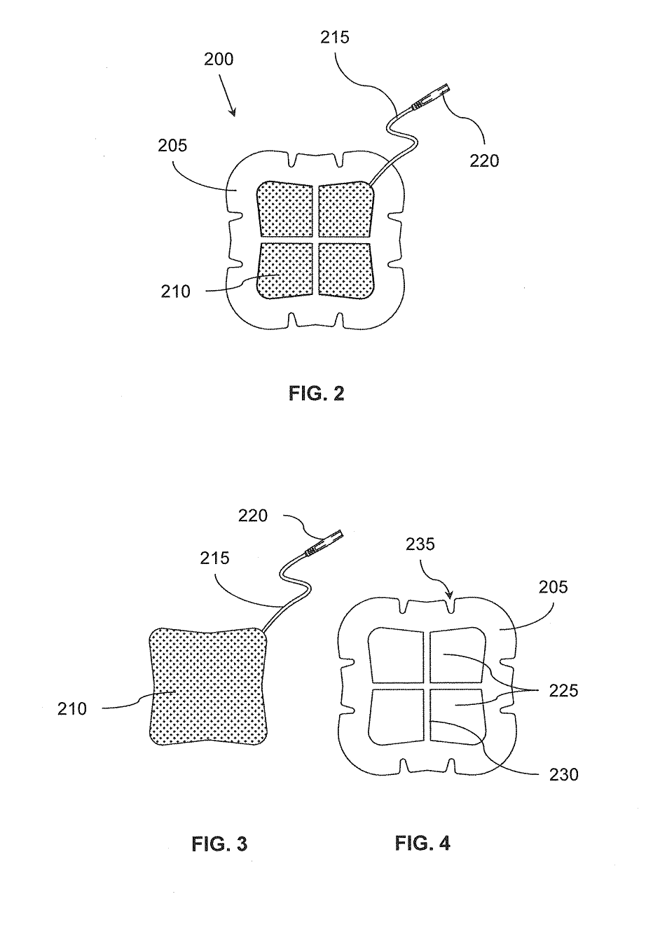

In another embodiment, with reference to FIG. 2, a transdermal device 200 is shown that includes a transdermal patch 205 and an electrode 210. In one embodiment, the electrode 210 is a self-adhesive electrode. For example, the patch 205 is configured to surround a perimeter of the self-adhesive electrode 210 to form a device that provides electrical stimulation (the electrode) and delivers a selected medicine/drug via the patch 205. As explained previously, the patch 205 is a substrate coated with a combined mixture of a non-water soluble adhesive and an anesthetic (e.g., lidocaine).

In this manner, the patch 205 includes material coated with the adhesive/anesthetic composition around the perimeter of the electrode 210. In another embodiment, the patch 205 and the electrode 210 may be reversed in their orientation (e.g., where the patch 205 is the central component and the electrode 210 is disposed around the perimeter of the patch 205). In either configuration, once the device 200 is attached to the skin of a patient, the anesthetic is applied to the skin from the material portions of the patch 205 while the electrode 210 may be controlled to apply electrical stimulation to an area beneath the skin that is in contact with the electrode 210.

With further reference to FIG. 2, the electrode 210 is made from an electrically conductive material and is electrically connected to an electrical wire 215 for supplying a current/voltage to the electrode 210. The wire 215 may include a connector or port 220 that is male or female in configuration for connecting to a power source. When the current/voltage is applied to the electrode 210, the electrode 210 generates electrical impulses that stimulate nearby tissue/muscle. In another embodiment, the electrode 210 is an ultrasound transducer that generates ultrasound energy.

In one embodiment, the patch 205 is removable from the electrode 210 or vice versa (e.g., separate components). FIG. 3 shows the electrode 210 separated from the patch 205 and FIG. 4 shows the patch 205 separated from the electrode 210. For example, the patch 205 and electrode 210 are attached together with an adhesive. In another embodiment, the patch 205 and the electrode 210 are integral with each other (e.g., a device with an electrode portion and a drug delivery portion).

With reference to FIGS. 3 and 4, in one embodiment, the patch 205 includes material defining an outer surface and a central opening 225. As seen in FIG. 2, the electrode 205 is exposed through the opening 225. The central opening 225 may be one opening through the patch 205 or may be multiple openings. As seen in FIG. 4, the opening 225 has four open areas defined by vertical and horizontal crossbars 230. Crossbars 230 may be included to provide support and stability to the sides of the patch 205 but are not required in all embodiments. In another embodiment, the patch material 205 may be solid without a central opening and the electrode 210 is attached to a portion of the patch 205.

With reference again to FIG. 2, in another embodiment, the electrode 210 may be configured without the wire 215 (e.g., wireless). For example, an electrical connector (not shown) may be attached directly on the surface of the electrode 210. As such, once the device 200 is attached to a patient, a power supply can be connected/attached to the connector on the electrode 205. In one embodiment, the connector may be a snap-on connector, a terminal, or other electrical connector.

In another embodiment, the electrode 210 surface is coated with the adhesive and anesthetic composition as described above. Thus the patch material 205 is eliminated. The electrode 210 functions as the transdermal delivery device.

### Definitions

The following includes definitions of selected terms employed herein.

The definitions include various examples and/or forms of components that fall within the scope of a term and that may be used for implementation. The examples are not intended to be limiting. Both singular and plural forms of terms may be within the definitions.

References to “one embodiment”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

While example systems, methods, and so on have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and so on described herein. Therefore, the disclosure is not limited to the specific details, the representative apparatus, and illustrative examples shown and described. Thus, this application is intended to embrace alterations, modifications, and variations that fall within the scope of the appended claims.

What is claimed is:

1. A transdermal delivery device comprising material coated on one surface with a mixture of a non-water soluble adhesive and an anesthetic.

2. The transdermal delivery device of claim 1, wherein the anesthetic is lidocaine and is combined with the non-water soluble adhesive.

3. The transdermal delivery device of claim 1, wherein the non-water soluble adhesive includes polyisobutylene.

4. The transdermal delivery device of claim 1, wherein the mixture includes a vasodilator composition.

5. The transdermal delivery device of claim 1, wherein the material forms a patch that is attachable to skin.

6. The transdermal delivery device of claim 1, wherein the material is configured as an adhesive bandage or tape.

7. The transdermal delivery device of claim 1, further comprising a self-adhesive electrode combined with the transdermal delivery device, wherein the self-adhesive electrode is attached to the material of the transdermal delivery device.

8. The transdermal delivery device of claim 7, wherein the material includes a central opening and wherein the self-adhesive electrode is attached and positioned within the central opening.
